(19) United States
(12) Patent Application Publication
(10) Pub. No.: US 2006/0128658 A1
(43) Pub. Date: Jun. 15, 2006

(54) Biocompatible dialysis fluids containing icodextrins

(76) Inventors: Leo Martis, Long Grove, IL (US); Carolyn Choo, Lincolnshire, IL (US); Paul Zieske, Glenview, IL (US)

Correspondence Address: BAXTER HEALTHCARE CORPORATION, 1 BAXTER PARKWAY, DF2-2E, DEERFIELD, IL, 60015, US

(21) Appl. No.: 11/351,922

(22) Filed: Feb. 10, 2006

Related U.S. Application Data
(63) Continuation of application No. 10/327,264, filed on Dec. 20, 2002, now abandoned.

(51) Int. Cl. A01N 1/02; A61K 31/19; A61K 31/715

(52) U.S. Cl. 514/58; 514/557; 435/1.1

(57) ABSTRACT

Icodextrin-based solutions and methods of making same that can be used during medical therapy, such as dialysis therapy are provided. The icodextrin-based solution at least includes a first solution containing icodextrin at a pH ranging from about 1.5 to about 5.0 and a buffer solution at a pH ranging from about 7.0 to about 12.0 that are so constructed and arranged allowing the icodextrin-based solution to be mixed prior to infusion into a patient. The icodextrin-based solutions of the present invention can be made at physiologic pH and with minimal glucose degradation products.

### CROSS REFERENCE TO RELATED APPLICATION

This patent application is a continuation of U.S. application Ser. No. 10/327,264 filed on Dec. 20, 2002, the disclosure of which is herein incorporated by reference.

### BACKGROUND

The present invention relates generally to medical treatments. More specifically, the present invention relates to fluids or solutions used for dialysis therapy.

Due to disease or insult or other causes, the renal system can fail. In renal failure of any cause, there are several physiological derangements. The balance of water, minerals (e.g., Na, K, Cl, Ca, P, Mg, SO₄) and the excretion of a daily metabolic load of fixed ions is no longer possible in renal failure. During renal failure, toxic end products of nitrogen metabolism (e.g., urea, creatinine, uric acid, and the like) can accumulate in blood and tissues.

Dialysis processes have been devised for the separation of elements in a solution by diffusion across a semi-permeable membrane (diffusive solute transport) across a concentration gradient. Examples of dialysis processes include hemodialysis, peritoneal dialysis and hemofiltration.

Hemodialysis treatment utilizes the patient's blood to remove waste, toxins, and excess water from the patient. The patient is connected to a hemodialysis machine and the patient's blood is pumped through the machine. Catheters are inserted into the patient's veins and arteries to connect the blood flow to and from the hemodialysis machine. Waste, toxins, and excess water are removed from the patient's blood and the blood is infused back into the patient. Hemodialysis treatments can last several hours and are generally performed in a treatment center about three or four times per week.

To overcome the disadvantages often associated with classical hemodialysis, other techniques were developed, such as hemofiltration and peritoneal dialysis. Hemofiltration is a convection-based blood cleansing technique. Blood access can be venovenous or arteriovenous. As blood flows through the hemofilter, a transmembrane pressure gradient between the blood compartment and the ultrafiltrate compartment causes plasma water to be filtered across the highly permeable membrane. As the water crosses the membrane, it convects small and large molecules across the membrane and thus cleanses the blood. An excessive amount of plasma water is eliminated by filtration. Therefore, in order to keep the body water balanced, fluid must be substituted continuously by a balanced electrolyte solution (replacement or substitution fluid) infused intravenously. This substitution fluid can be infused either into the arterial blood line leading to the hemofilter (predilution) or into the venous blood line leaving the hemofilter.

Peritoneal dialysis utilizes the patient's own peritoneum as a semipermeable membrane. The peritoneum is the membranous lining of the body cavity that, due to the large number of blood vessels and capillaries, is capable of acting as a natural semipermeable membrane.

In peritoneal dialysis, a sterile dialysis solution is introduced into the peritoneal cavity utilizing a catheter. After a sufficient period of time, an exchange of solutes between the dialysate and the blood is achieved. Fluid removal is achieved by providing a suitable osmotic gradient from the blood to the dialysate to permit water outflow from the blood. This allows a proper acid-base, electrolyte and fluid balance to be returned to the blood. The dialysis solution is simply drained from the body cavity through the catheter. Examples of different types of peritoneal dialysis include continuous ambulatory peritoneal dialysis, automated peritoneal dialysis and continuous flow peritoneal dialysis.

Standard peritoneal dialysis solutions contain dextrose at a concentration of 1.5% to 4.25% by weight to effect transport of water and metabolic waste products across the peritoneum. Although dextrose has the advantage of being relatively safe and inexpensive, it has a number of disadvantages. Because of the small size, dextrose is rapidly transported through the peritoneum, thus leading to the loss of osmotic gradient and loss of ultrafiltration within about 2 to 4 hours of infusion. It has been suggested that the ultrafiltration characteristics of peritoneal dialysis solutions could be improved by replacing dextrose with large molecular weight substances, such as icodextrin. Dialysis solutions containing icodextrin are commercially available and have been found to be useful in treating patients with end stage renal disease.

Like dextrose, glucose polymers are not stable during terminal heat sterilization (a pharmacoepial requirement for peritoneal dialysis fluids) if they are formulated at physiologic pH. As a result, icodextrin containing solutions are typically formulated at an acid pH, such as a pH between 5.0 to 5.5. However, the low pH can cause pain on infusion in some patients and is cytotoxic to peritoneal cells including mesothelial cells, macrophages and fibroblasts. In addition, even at pH 5.0 to 5.5, icodextrin can undergo degradation, thus resulting in a wide variety of degradation products that can lead to the formation of advanced glycation end products (AGEs). AGEs are believed to damage the peritoneal membrane and end of peritoneal dialysis to sustain life in kidney disease patients.

Therefore, a need exists to provide improved medical solutions that can be readily manufactured, that can remain stable and sterile under storage conditions, and that can be readily and effectively used during medical therapy, such as dialysis therapy.

### SUMMARY

The present invention relates to improved icodextrin-based solutions and methods of making same that can be used during medical therapy, such as dialysis therapy. The icodextrin-based solutions of the present invention can be made at physiologic pH and with minimal glucose degradation products. This provides improved biocompatibility, particularly as applied during peritoneal dialysis.

In an embodiment, the present invention provides a solution that at least includes a first solution containing an icodextrin at a pH ranging from about 1.5 to about 5.0 and a buffer solution at a pH ranging from about 7.0 to about 12.0 wherein the first part and the second part are so constructed and arranged that the first part and the second part are mixed prior to infusion into a patient. For example, the first part can be stored in a first chamber of a multi-chamber container and the buffer solution can be stored in a second chamber of a multi-chamber container prior to mixing and infusion into a patient during peritoneal dialysis. By way of further example, the solutions can be provided separately as concentrates and a mixing device, such as the BAXTER HOMECHOICE®, can be used to mix the solution immediately prior to infusion.

The first solution is acidified with an acid, such as an organic acid (e.g., lactic acid, acetic acid, pyruvatic and all of the intermediates of the KREBS tri-carboxylic acid cycle), an inorganic acid (e.g., hydrochloric acid), the like and combinations thereof. Further, the first solution includes about 100.0 to about 220.0 (g/L) of icodextrin and other components, such as calcium chloride, magnesium chloride, calcium chloride dihydrate, magnesium chloride hexahydrate, the like and combinations thereof. The buffer solution includes one or more components, such as sodium chloride, sodium lactate, sodium bicarbonate, one or more amino acids with a pK1 between 7 and 13, such as histidine, glycine, alanine, etc., the like and combinations thereof.

When mixed, the first part and the second part can form a mixed solution which includes, for example, about 4.0 to about 10.0 (g/dL) of icodextrin; about 0.5 to about 4.0 (mEq/L) of calcium; about 0.25 to about 2.0 (mEq/L) of magnesium; about 120.0 to about 135.0 (mEq/L) of sodium; about 90.0 to about 110.0 (mEq/L) of chloride; about 30.0 to about 45.0 (mEq/L) of lactate and the like. The mixed solution can further include, for example, about 5.0 mM or less of bicarbonate, about 5.0 mM or less of histidine, the like and combinations thereof.

In an embodiment, the peritoneal dialysis solution of the present invention has a pH ranging from about 6.5 to about 7.4. A volume ratio of the icodextrin-based solution to the buffer solution can include about 3:1 to about 1:3.

In another embodiment, the present invention provides a method of producing a peritoneal dialysis solution. The method includes preparing a first solution and a buffer solution wherein the first solution includes icodextrin at a pH ranging from about 1.5 to about 5.0 and wherein the buffer solution has a pH ranging from about 7.0 to about 12.0; and mixing the first solution and the buffer solution prior to infusion into a patient.

In yet another embodiment, the present invention provides a method of providing dialysis therapy to a patient. The method includes the preparation of a first solution and a buffer solution wherein the first solution includes icodextrin at pH ranging from about 1.5 to about 5.0 and wherein the buffer solution has a pH ranging from about 7.0 to about 12.0; mixing at least the first solution and the buffer solution to form a mixed solution; and infusing the mixed solution into the patient.

In still yet another embodiment, the peritoneal dialysis solution of the present invention has a first part including a first solution containing icodextrin, calcium, and magnesium wherein the first part has a pH ranging from about 2.5 to about 5.0; and a second part that includes sodium chloride and sodium lactate and has a pH of about 7 to about 12. The first part and the second part are so constructed and arranged that the first part and the second part are mixed to form a mixed solution prior to infusion into a patient wherein the mixed solution has a pH ranging from about 6.5 to about 7.4.

An advantage of the present invention is to provide improved peritoneal dialysis solutions.

Another advantage of the present invention is to provide peritoneal dialysis solutions which can be made at physiologic pH.

Furthermore, an advantage of the present invention is to provide peritoneal dialysis solutions with minimal glucose degradation products.

Moreover, an advantage of the present invention is to provide improved icodextrin-based solutions.

Another advantage of the present invention is to provide icodextrin-based solutions that can be effectively used during dialysis therapy, such as peritoneal dialysis.

Still another advantage of the present invention is to provide improved methods for producing improved solutions at least containing icodextrins at physiologic pH.

Yet another advantage of the present invention is to provide medical therapies, such as dialysis therapy, that employ the use of a ready to use and stable icodextrin-based solutions.

Additional features and advantages of the present invention are described in, and will be apparent from, the following Detailed Description of the Invention and the FIGURES.

### BRIEF DESCRIPTION OF THE FIGURES

FIG. 1 illustrates an icodextrin-based solution stored in a container pursuant to an embodiment of the present invention.

### DETAILED DESCRIPTION

The present invention provides improved peritoneal dialysis solutions as well as methods of manufacturing and using same. More specifically, the present invention relates to icodextrin-based solutions that can be used as a part of dialysis therapy and are provided as ready to use and stable solutions. As previously discussed, the icodextrin-based solutions of the present invention can be made at physiologic pH and with minimal glucose degradation products. This provides improved biocompatibility, particularly as applied during dialysis therapy, such as peritoneal dialysis.

With respect to dialysis therapy, the present invention can be used in a variety of different dialysis therapies to treat kidney failure. Dialysis therapy as the term or like terms are used throughout the text is meant to include and encompass any and all forms of therapies that utilize the patient's blood to remove waste, toxins and excess water from the patient. Such therapies, such as hemodialysis, hemofiltration and hemodiafiltration, include both intermittent therapies and continuous therapies used for continuous renal replacement therapy (CRRT). The continuous therapies include, for example, slow continuous ultrafiltration (SCUF), continuous venovenous hemofiltration (CVVH), continuous venovenous hemodialysis (CVVHD), continuous venovenous hemodiafiltration (CVVHDF), continuous arteriovenous hemofiltration (CAVH), continuous arteriovenous hemodialysis (CAVHD), continuous arteriovenous hemodiafiltration (CAVHDF), continuous ultrafiltration periodic intermittent hemodialysis or the like. The icodextrin-based solutions can also be used during peritoneal dialysis including, for example, continuous ambulatory peritoneal dialysis, automated peritoneal dialysis, continuous flow peritoneal dialysis and the like. Further, although the present invention, in an embodiment, can be utilized in methods providing a dialysis therapy for patients having chronic kidney failure or disease, it should be appreciated that the present invention can be used for acute dialysis needs, for example, in an emergency room setting. Lastly, as one of skill in the art appreciates, the intermittent forms of therapy (i.e., hemofiltration, hemodialysis, peritoneal dialysis and hemodiafiltration) may be used in the in center, self/limited care as well as the home settings.

In an embodiment, the icodextrin-based solution can be used as a dialysate during any suitable dialysis therapy. Alternatively, the solutions of the present invention can be administered or infused into a patient as a replacement solution, infusion solution or the like during dialysis therapy, particularly during continuous renal replacement therapy. In this regard, replacement solutions, infusion solutions or the like must necessarily be continuously fed to a patient as a substitute for an excessive amount of plasma water that is typically removed during continuous renal replacement therapy. In this regard, a proper water balance in the patient's body can be effectively maintained.

The icodextrin-based solutions of the present invention can include a variety of different components in any suitable amount. The solution at least includes two parts that are mixed prior to use. For example, the first part includes a first solution containing an icodextrin. In an embodiment, the icodextrin is in an amount ranging from about 100.0 g/L to about 220.0 g/L. Further, the first part has a pH ranging from about 1.5 to about 5.0, such as 2.5, 3.0 and the like. In this regard, degradation of the icodextrin-based solution can be minimized during heat sterilization. It should be appreciated that the icodextrin-based solution can be sterilized in any suitable way, such as filtration sterilization, heat sterilization, steam sterilization, radiation sterilization and/or like sterilization techniques.

The first part can include a number of suitable and different types and amounts of components in addition to icodextrin. For example, the first part includes an acid, such as an organic acid (e.g., lactic acid, acetic acid, pyruvatic acid and all of the intermediates of the KREBS tri-carboxylic acid cycle), an inorganic acid (e.g., hydrochloric acid), the like and combinations thereof. In an embodiment, the first solution includes about 100.0 to about 220.0 (g/L) of icodextrin, about 5.0 to about 10.0 (mEq/L) of calcium chloride dihydrate, about 0.5 to about 2.0 (mEq/L) of magnesium chloride hexahydrate, the like and combinations thereof.

The second part can include a variety of different and suitable materials. In an embodiment, the second part of the icodextrin-based solution includes a buffer solution at a pH ranging from about 7.0 to about 12.0. The buffer solution can include, for example, sodium bicarbonate, sodium chloride, sodium lactate, one or more amino acids with a pK1 between 7 and 13, such as histidine, glycine, alanine, etc., the like and combinations thereof.

It should be appreciated that the icodextrin-based solutions of the present invention can include any suitable type, number and amount of additional components. For example, the solutions of the present invention can include one or more of any suitable type and amount of small molecular weight osmotic agents, such as glucose, glycerol, amino acids, peptides, the like and combinations thereof. The small molecular weight osmotic agents of the first part can include, for example, glucose, glycerol and/or the like. In an embodiment, the small molecular weight osmotic agent concentration of the first part ranges from about 1% to about 6%. The small molecular weight osmotic agents of the second part can include, for example, amino acids, peptides and/or the like. In an embodiment, the small molecular weight osmotic agent concentration of the second part ranges from about 1% to about 6%. When the first part and the second part are mixed and combined to form the icodextrin-based solution of the present invention, the small molecular weight osmotic agent concentration of the icodextrin-based solution, in an embodiment, ranges from about 0.5% to about 4%.

The pH can be adjusted to include any suitable pH within the pH range as discussed above. For example, the pH can be adjusted to about 7.0 to about 9.0, preferably to about 7.0 to about 8.0, using a pH stabilizer, such as sodium bicarbonate, histidine, the like and combinations thereof. In an embodiment, the pH of the buffer chamber can range from about 9.0 to about 12.0. This pH range can be effectively used when lactate is substituted with bicarbonate so that bicarbonate exists as carbonate. This would eliminate the need for a gas barrier overpouch to contain CO₂ within the solution.

In an embodiment, the first part and the second part are so constructed and arranged that at least the first part and the second part are mixed prior to infusion into a patient. For example, the first part is stored in a first chamber of a multi-chamber container and the second part is stored in a second chamber of the multi-chamber container.

It should be appreciated that the components of the solution can be housed or contained in any suitable manner such that the icodextrin-based solutions of the present invention can be effectively prepared and administered. In an embodiment, the present invention includes a two part icodextrin-containing solution in which each part or component are formulated and stored separately, and then mixed just prior to use. A variety of containers can be used to house the two part icodextrin-containing solution, such as separate containers (i.e., flasks or bags) that are connected by a suitable fluid communication mechanism. In an embodiment, a multi-chamber container or bag can be used to house the separate components of the solution as previously discussed. By way of further example, the solutions can be provided separately as concentrates and a mixing device, such as the BAXTER HOMECHOICE®, can be used to mix the solutions immediately prior to infusion.

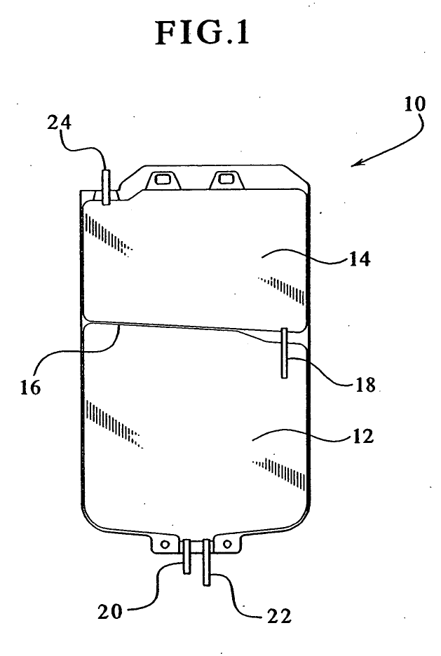

FIG. 1 illustrates a suitable container for storing, formulating and administering a bicarbonate-based solution of the present invention. The multi-chamber bag 10 has a first chamber 12 and a second chamber 14. The interior of the container is divided by a heat seal 16 into two chambers. It should be appreciated that the container can be divided into separate chambers by any suitable seal. In an embodiment, the container can be divided into separate chambers, such as two chambers, by a peel seal. The multi-chamber container 10 also has a frangible connector 18 to sealingly couple the first chamber 12 to the second chamber 14. To mix the solution within the multi-chamber bag 10, the frangible connector 18 is broken.

The first container or chamber 12 includes two port tubes having, for example, different lengths. As shown in FIG. 1, the short port tube 20 can be utilized to add other constituents to the first chamber 12 during formulation of the solution of the present invention, if necessary. The long port tube 22 can be utilized to adaptedly couple the first chamber 12 to the patient via, for example, a patient's administration line (not shown). The second container or chamber 14 has a single port tube 24 extending therefrom which is closed by, for example, a solid rod (not shown). In this regard, it is not possible to add any additional constituents to this chamber and/or connect this chamber to a patient's administration line such that the chamber 14 cannot be adapted to deliver its constituents to the patient.

In an embodiment, the transfer of product within the multi-chamber bag 10 is thereby initiated from the second chamber 14 to the first chamber 12 such that the components of each chamber can be properly mixed to form the icodextrin-based solution of the present invention. In this regard, the first chamber 12 is larger in volume than the second chamber 14 such that the components of each chamber can be properly mixed once the transfer from the second chamber to the first chamber has occurred. Thus, the multi-chamber bag 10 can house at least two solutions that after mixture will result in a ready-to-use dialysis solution. An example of the multi-chamber container is set forth in U.S. Pat. No. 5,431,496, the disclosure of which is incorporated herein by reference. The multi-chamber bag can be made from a gas permeable material, such as polypropylene, polyvinyl chloride or the like.

In an embodiment, the container can be made with a gas barrier in any suitable way. For example, the gas barrier can be in the container material. Alternatively, the gas barrier can be an over pouch, a secondary liner or the like. The gas barrier can be composed of any suitable materials. In an embodiment, the gas barrier is composed of ethylvinyl acetate, polyvinyl dichloride, a copolymer of ethylvinyl acetate and polyvinyl dichloride, other suitable materials including polymeric materials and combinations thereof.

It should be appreciated that the container of the present invention can be manufactured from a variety of different and suitable materials and configured in a number of suitable ways such that the icodextrin-based solution of the present invention can be effectively formulated and administered to the patient during medical therapy. For example, the second chamber can be larger in volume than the first chamber such that the icodextrin-based solution of the present invention can be readily and effectively made and administered to the patient from the second chamber.

The icodextrin-based solution can be prepared by mixing at least two parts prior to use. In an embodiment, the mixed icodextrin-based solution of the present invention at least includes about 4.0 to about 10.0 (g/dL) of icodextrin, about 0.5 to about 4.0 (mEq/L) of calcium, about 0.25 to about 2.0 (mEq/L) of magnesium, about 120.0 to about 135.0 (mEq/L) of sodium, about 90.0 to about 110.0 (mEq/L) of chloride, about 30.0 to about 45.0 (mEq/L) of lactate, the like and combinations thereof. For example, the mixed solution can include about 5.0 mM or less of bicarbonate, about 5.0 mM or less of histidine and combinations thereof.

In an embodiment, the mixed solution has a pH ranging from about 6.5 to about 7.4. The pH stabilizer of the second part can be included in the mixed solution, in an embodiment, in an amount ranging from about 25.0 mEq/L to about 45.0 mEq/L. The icodextrin-based solution includes, in an embodiment, a volume ratio of the icodextrin-containing solution and the buffer solution that ranges from about 3:1 to about 1:3.

By way of example and not limitation examples of the present invention will now be set forth.

### COMPOSITION EXAMPLE ONE

COMPOSITION IN ICODEXTRIN CHAMBER

| | |
|---|---|
| Icodextrin (g/L) | 100.0-220.0 |
| Calcium Chloride dihydrate (mEq/L) | 5.0-10.0 |
| Magnesium Chloride hexahydrate (mEq/L) | 0.5-2.0 |

HCl for pH adjustment between 2.5 and 5.0

COMPOSITION OF THE BUFFER CHAMBER

| | |
|---|---|
| Sodium Chloride (mEq/L) | 50.0-150.0 |
| Sodium Lactate (mEq/L) | 50.0-120.0 |

Sodium Bicarbonate and/or Histidine for pH adjustment
between 8.0 and 9.0

### COMPOSITION EXAMPLE TWO

COMPOSITION IN ICODEXTRIN CHAMBER (Large Chamber)

| | |
|---|---|
| Icodextrin (g/L) | 121 |
| Sodium Chloride (g/L) | 4.22 |
| Calcium Chloride Dihydrate (g/L) | 0.40 |
| Magnesium Chloride Hexahydrate (g/L) | 0.08 |
| Sodium Lactate (g/L) | 3.50 |
| pH | about 5.0 to about 5.4 |

COMPOSITION IN BUFFER CHAMBER (Small Chamber)

| | |
|---|---|
| Sodium Chloride (g/L) | 7.42 |
| Sodium Lactate (g/L) | 6.15 |
| Sodium Bicarbonate (g/L) | 0.58 |
| pH | about 8.2 to about 8.7 |

ICODEXTRIN AND IONIC COMPOSITION
OF THE MIXED SOLUTION

| | |
|---|---|
| Icodextrin (g/dL) | 4.0-10.0 |
| Calcium (mEq/L) | 0.5-4.0 |
| Magnesium (mEq/L) | 0.25-2.0 |
| Sodium (mEq/L) | 120.0-135.0 |
| Chloride (mEq/L) | 90.0-110.0 |
| Lactate (mEq/L) | 30.0-45.0 |
| Bicarbonate or Histidine (mM) | NMT 5.0 |

As used herein, the term “NMT” means not more than.

ICODEXTRIN CHARACTERISTICS

| | |
|---|---|
| Weight Average Molecular Weight | 10,000-20,000 |
| Number Average Molecular weight | 4,000-8,000 |
| Polydispersity | 1.0-4.0 |
| Fraction >100,000 | NMT 1.0% |
| Mono, Di, Tri-Saccharides | NMT 5.0% |
| Linear Polymers (alpha 1,4) | NLT 90.0% |
| Branched Polymers (alpha 1,6) | NMT 10.0% |
| Aluminum (10% solution) | <10 ppb |
| Aqueous Solubility | NLT 22.0% |
| Heavy Metals | <5 ppm |

As used herein, the term “NLT” means not less than.

DEGREE OF POLYMERIZATION OF ICODEXTRIN (DP)

| | |
|---|---|
| DP greater than 20 | >75% |
| DP greater than 40 | >50% |
| DP greater than 80 | >25% |

### Experiment One

This experiment was performed to determine the effect of pH on the stability of icodextrin (7.5% solution). Stability of icodextrin was assessed by measuring the absorbency of icodextrin solution at different pH values before and after sterilization:

| Pre-sterilization | Post-sterilization | | |
|---|---|---|---|
| (pH) | (pH) | AU 284 nm | AU 228 nm |
| 5.5* | 5.4 | 0.022 | 0.044 |
| 4.0 | 3.9 | 0.011 | 0.012 |
| 3.5 | 3.5 | 0.013 | 0.010 |
| 3.0 | 3.0 | 0.011 | 0.010 |
| 2.5 | 2.5 | 0.016 | 0.014 |

*This was a commercially available icodextrin solution. The remaining solutions tested pursuant to EXPERIMENT ONE were prepared according to an embodiment of the present invention.

The data of EXPERIMENT ONE suggest that the degradation of icodextrin could be reduced by more than 50% by adjusting pre-sterilization pH between 2.5 and 4.0. It is noted that too acidic of a pH results in hydrolysis of icodextrin that results in a change of the molecular weight of the icodextrin. The optimum pH of the icodextrin chamber is where hydrolysis and degradation are minimal.

### Experiment Two

This experiment was performed to determine the pH of the mixed solution that was prepared according to an embodiment of the present invention.

Part One solution was prepared by mixing the following components in 1 liter of solution:

| | | |
|---|---|---|
| Icodextrin | 207 | gms |
| Calcium chloride dehydrate | 0.710 | gms |
| Magnesium chloride hexahydrate | 0.140 | gms |
| HCl added to adjust the pH to 3.0 | | |
| Solution volume | 758 | mL |

Part Two solution was prepared by mixing following components in 1 liter of solution:

| | | |
|---|---|---|
| Sodium chloride | 8.44 | gms |
| Sodium lactate | 7.03 | gms |
| Sodium bicarbonate added to adjust the pH to 8.3 | | |
| Solution volume | 1332 | ml |

The Part One and Part Two solutions were combined to form a mixed solution with the following composition:

| | | |
|---|---|---|
| Icodextrin | 7.5 | gm/dL |
| Calcium | 3.5 | mEq/L |
| Magnesium | 0.5 | mEq/L |
| Sodium | 132 | mEq/L |
| Chloride | 96 | mEq/L |
| Lactate | 40 | mEq/L |

| | |
|---|---|
| pH | 7.0 |

The results of EXPERIMENT TWO indicate that the two part solution prepared as discussed above pursuant to an embodiment of the present invention has a composition that is ideal for use in peritoneal dialysis. The two part solution and the use of pH adjustor in a manner described above pursuant to an embodiment of the present invention provides icodextrin-based solutions that can be prepared with improved stability, pH and thus enhanced biocompatibility.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A peritoneal dialysis solution comprising:
a first part including a first solution including a glucose polymer and lactate; and
a second part including a buffer solution comprising lactate.

2. The peritonal dialysis solution of claim 1, wherein the first part includes a glucose polymer in an amount ranging from about 100.0 g/L to about 220.0 g/L.

3. The peritoneal dialysis solution of claim 1, wherein the glucose polymer includes an icodextrin.

4. The peritoneal dialysis solution of claim 1, wherein the pH of the first solution is not greater than 5.0

5. An icodextrin-based solution comprising:
a first part including an icodextrin and lactate; and
a second part including a buffer solution comprising bicarbonate.

6. The icodextrin-based solution of claim 5, wherein the first part includes icodextrin in an amount ranging from about 100.0 g/L to about 220.0 g/L.

7. The icodextrin-based solution of claim 5, wherein the buffer solution includes a pH ranging from about 7.0 to about 12.0.

8. The icodextrin-based solution of claim 5, wherein the pH of the first part is not greater than 5.0

9. A peritoneal dialysis solution comprising:
a first solution including a glucose polymer and lactate;
a buffer solution comprising lactate and bicarbonate; and
the first solution and the buffer solution being mixed prior to infusion into a patient to create a resultant solution including bicarbonate at no greater than 5 mmol/L.

10. The peritoneal dialysis solution of claim 9, wherein the first part includes the glucose polymer in an amount ranging from about 100.0 g/L to about 220.0 g/L.

11. The peritoneal dialysis solution of claim 9, wherein the glucose polymer includes an icodextrin.

12. A peritoneal dialysis solution comprising:
a first solution part including a glucose polymer, lactate and an inorganic acid; and
a second solution part including a buffer comprising lactate.

13. The peritoneal dialysis solution of claim 12, wherein the glucose polymer includes an icodextrin.

14. The peritoneal dialysis solution of claim 12, wherein the first part includes the glucose polymer in an amount ranging from about 100.0 g/L to about 220.0 g/L.

15. A peritoneal dialysis solution comprising:
a first solution including a glucose polymer and lactate;
a buffer solution comprising lactate; and
the first solution and the buffer solution being mixed prior to infusion into a patient to create a resultant peritoneal dialysis solution that does not include an amino acid.

16. The peritoneal dialysis solution of claim 15, wherein the glucose polymer includes icodextrin in an amount ranging from about 100.0 g/L to about 220.0 g/L.

17. A peritoneal dialysis solution comprising:
a first solution including a glucose polymer, lactate and a pH adjustment agent not including an organic acid, the first solution having a pH not greater than 5.0;
a buffer solution comprising lactate; and
the first solution and the buffer solution being mixed prior to infusion into a patient to create a resultant peritoneal dialysis solution that does not include an amino acid.

18. The peritoneal dialysis solution of claim 17, wherein the glucose polymer includes icodextrin in an amount ranging from about 100.0 g/L to about 220.0 g/L.

19. The peritoneal solution of claim 17, wherein the buffer solution includes a pH ranging from about 7.0 to about 12.0.

20. The peritoneal dialysis solution of claim 17, wherein the pH of the first solution is not greater than 5.0.
